(19) United States
(12) Patent Application Publication
(10) Pub. No.: US 2006/0166956 A1
(43) Pub. Date: Jul. 27, 2006

(54) Use of anecortave acetate for the protection of visual acuity in patients with age related macular degeneration

(76) Inventors: Janice A. Jerdan, Arlington, TX (US); Patricia Zilliox, Arlington, TX (US); Stella M. Robertson, Forth Worth, TX (US)

Correspondence Address: Alcon Research, 6201 South Freeway, Fort Worth, TX, 76134-2099, US

(21) Appl. No.: 10/521,707

(22) PCT Filed: Jun. 26, 2003
(86) PCT No.: PCT/US03/20154

§ 371 Date: Jan. 19, 2005

Related U.S. Application Data
(60) Provisional application No. 60/401,220, filed on Aug. 5, 2002.

(51) Int. Cl. A61K 31/57

(52) U.S. Cl. 514/178

(57) ABSTRACT

The present invention is directed to the use of anercortave acetate or the alcohol thereof for the protection of visual acuity in patients with age related macular degeneration.

This application claims priority from U.S. Ser. No. 60/401,220, filed Aug. 5, 2002.

The present application is directed to the use of anecortave acetate to maintain vision and provide protection of visual acuity in patients with age related macular degeneration (AMD).

### BACKGROUND OF THE INVENTION

AMD is currently the primary cause of functional blindness in patients over the age of 50 in developed countries. Although the exudative form is present in only 15-20% of the AMD population, exudative AMD accounts for much of the significant vision loss (1). Until recently, the only approved treatment for CNV associated with exudative AMD was laser photocoagulation. In 2000, photodynamic therapy with Visudyne® was approved for the treatment of selected subfoveal lesions in this patient population. However, this treatment option has been shown to delay, but not stop, loss of vision in a great majority of the patients treated (2).

Because irreversible retinal damage due to exudative AMD is the direct result of abnormal choroidal blood vessel growth beneath the retina and/or the retinal pigment epithelium (RPE), a number of angiostatic agents are now being evaluated clinically for use in treating this blinding disorder. Angiogenesis is a complex of inter-related processes with numerous potential opportunities for therapeutic intervention. In contrast to other experimental therapies for AMD, which were designed to specifically inhibit angiogenesis stimulated by vascular endothelial growth factor (VEGF) (3,4), anecortave acetate inhibits blood vessel growth by inhibiting the proteases necessary for vascular endothelial cell migration (5,6). Anecortave acetate is unique in that it inhibits angiogenesis subsequent to (and therefore independently of) the actual angiogenic stimulus, and it therefore has the potential to nonspecifically inhibit angiogenesis driven by the wide variety of known ocular angiogenic stimuli (7). The ability of anecortave acetate to inhibit angiogenesis independently of the initiating stimulus is supported by a large body of preclinical evidence, including multiple animal models of neovascularization (6, 8-10).

### SUMMARY OF THE INVENTION

The present invention is directed to preparations and methods for the prevention of the loss of visual acuity associated with AMD, the maintenance of visual acuity in persons suffering from AMD, and the inhibition of lesion growth associated with AMD. The preparations and methods involve 3-30 mg. of anecortave acetate or its corresponding alcohol administered juxtasclerally providing for transcleral delivery of the drug.

### BRIEF DESCRIPTION OF THE DRAWINGS

Figure Legends:

Table 1. Eligibility criteria for patient enrollment in this study

Table 2. Baseline characteristics of patients enrolled in this study. No significant differences between treatment groups were identified for any parameter.

Table 3. Log MAR visual acuity changes from Baseline at Month 6 across treatment groups expressed as log MAR lines of worsening or improvement. There is a clear trend favoring treatment with a single administration of anecortave acetate 15 mg over placebo treatment for prevention of clinically significant vision loss, defined as a worsening by ≧3 log MAR lines of vision or 15 log MAR letters (12% versus 30%) from Baseline.

Table 4. Analysis of severe vision loss at Month 6 compared with Baseline among treatment groups. Treatment with Anecortave acetate 15 mg is statistically superior to placebo treatment (p=0.0224) for prevention of severe vision loss, defined as a worsening by ≧6 log MAR lines of vision or 30 log MAR letters.

FIG. 1. When mean change from Baseline log MAR visual acuity at Month 6 is compared among treatment groups, there is a statistically significant difference (p=0.0032) between treatment with anecortave acetate 15 mg and placebo treatment. After a single treatment of anecortave acetate 15 mg, mean log MAR vision changes at Month 6 by less than 1 line (by 4 log MAR letters) to a +0.08 log MAR score. In contrast, after a single placebo treatment the mean log MAR vision had worsened by more than 2 lines (by 12 log MAR letters) to a +0.24 log MAR score over the same period. This difference in Month 6 mean log MAR scores is statistically significant (p=0.0032)

FIG. 2. Comparison of all 128 patients in the four treatment groups as to preservation of vision at Month 6, defined as a decrease of less than 3 log MAR lines or 15 log MAR letters from Baseline values. Although statistical significance is not achieved in this analysis, there is a clear trend favoring the anecortave acetate 15 mg treatment group over placebo treatment.

FIG. 3. Subgroup analysis comparing the effect of the four treatment groups for preservation of vision at Month 6 in patients with predominantly classic lesions at Baseline. There is statistical significance (p=0.0209) when the anecortave acetate 15 mg treatment group is compared with placebo treatment for this large subgroup of patients.

FIG. 4. The percentage of patients with improved vision, defined as an increase of at least 2 log MAR lines or 10 log MAR letters of visual acuity at Month 6 compared with Baseline values. This overall analysis of all 128 patients enrolled in the study reveals a statistically significant positive effect of anecortave acetate 15 mg for improvement of vision at Month 6 (p=0.025) compared with placebo.

FIG. 5. An overall analysis of the percent change in lesion growth at Month 6 compared with Baseline. There is a statistically significant positive effect of anecortave acetate 15 mg compared with placebo for inhibition of the total lesion growth (p=0.0005), the total CNV component (p=0.0001), and the classic CNV component (p=0.0008).

### DETAILED DESCRIPTION OF THE INVENTION

Anecortave acetate (4,9(11)-pregnadien-17α,21-diol-3,20 dione-21 acetate) is being clinically evaluated as monotherapy to treat exudative subfoveal AMD in this ongoing multi-center trial. The results of an interim analysis of the first 6 months of clinical data on safety and efficacy following a single treatment are reported here.

This ongoing trial was initiated to compare the clinical efficacy of anecortave acetate versus placebo treatment for preservation (maintenance) of vision and inhibition of CNV lesion growth. Patients with a log MAR visual acuity of 0.3 (20/40 Snellen equivalent) to 1.2 (20/320 Snellen equivalent) and primary or recurrent subfoveal choroidal neovascularization (CNV) secondary to AMD with a lesion up to 30.48 mm² (12 disc areas) in size were enrolled. Inclusion and exclusion criteria for this study are listed in Table 1. At Baseline and follow-up visits, best-corrected log MAR visual acuity was obtained on all patients using guidelines previously established for the Early Treatment Diabetic Retinopathy Study. Patient lesion eligibility for this study was determined from standardized fluorescein angiograms at the Digital Angiography Reading Center (DARC) by certified Readers (trained retina specialists) prior to enrollment and treatment. The DARC also evaluated changes from Baseline in the fluorescein angiographic characteristics of the lesions in masked fashion. Each data point represents the average of at least two independent evaluations by DARC Readers. Because all angiographic data for this study is being collected using the same fundus camera and digital camera systems and stored as uncompressed digital images, the actual lesion surface areas can be more closely approximated in mm² rather than requiring the disc area “best-fit” estimates previously used for film angiographic data.

The 128 patients in this double-masked, dose-response study were enrolled and treated between April 1999 and May 2001 by 18 participating sites in the US and EU. Prior to treatment, patients were enrolled and equally randomized to anecortave acetate sterile suspension for injection 30 mg (N=33), 15 mg (N=33), or 3 mg (N=32) or to placebo (vehicle, N=30). Masking of the clinical sites as to treatment group is being maintained in two ways. Study medication is masked by placing the treatment kits including study medication and supplies for the posterior juxtascleral administration in sealed opaque boxes identified by patient number only. The boxes were numbered sequentially at each clinical site and patients were assigned the next available sequential number upon enrollment. The randomization was built into the sequential numbering of the treatment kits and blocked within each site to maintain equal distribution across treatment assignments. Masking as to treatment group is also being maintained at each site by having an unmasked injecting investigator perform the treatments and a masked examining investigator perform the subsequent evaluations. Upon enrollment of each patient, anecortave acetate or placebo was administered behind the eye as a 0.5 mL posterior juxtascleral injection onto the outer surface of the sclera near the macula using a specially designed cannula. The cannula is described in commonly owned U.S. Pat. No. 6,413,245B 1.

Clinical efficacy data is being obtained from evaluations of best-corrected log MAR visual acuity and standardized fluorescein angiograms. Clinical safety data obtained from general physical examinations, laboratory evaluations of blood and urine, and complete ophthalmic examinations, including indocyanine green angiography, continue to be periodically evaluated by the Independent Safety Committee overseeing this study. Clinical data from evaluations for safety and efficacy performed at Day 1-2, Week 2, Week 6, Month 3, and Month 6 following patient randomization and treatment are reported here.

The primary efficacy outcome for this ongoing study is the mean change from Baseline in best-corrected log MAR visual acuity. Secondary efficacy outcomes are: the percentage of patients with preservation or maintenance of vision (defined as loss of less than three log MAR lines [less than 15 log MAR letters] of visual acuity); the percentage of patients with clinically significant worsening of vision (defined as a loss of at least three log MAR lines [at least 15 log MAR letters] of visual acuity); the percentage of patients with severe vision loss (defined as a loss of at least six log MAR lines [at least 30 log MAR letters] of visual acuity); and changes in CNV lesion characteristics (defined as total lesion area, total CNV and total classic CNV).

All efficacy analyses were based on the intent-to-treat principle. All patients received the medication to which they were assigned, and were analyzed accordingly. Last-observation-carry-forward was used to impute missing values. Baseline comparisons were tested using analysis of variance (for continuous outcomes) and Pearson's chi square (for binary outcomes). Changes from baseline in visual acuity and lesion parameters were tested in a repeated measures analysis of variance model with appropriate contrasts for the 6-month comparisons. Comparisons of binary outcomes at Month 6 were evaluated using Pearson's chi-square tests. All analyses of ocular outcomes are based on changes in the study (i.e., treated) eye.

In this ongoing study, re-treatment with study medication is being performed by the unmasked injecting investigator if the masked examining investigator judges that the patient may benefit. A 6-month re-treatment interval was established for this ongoing study based upon preclinical data demonstrating that anecortave acetate administered as a slow-release depot adjacent to the posterior scleral surface provided therapeutic drug levels in the adjacent choroid and retina for up to 6 months (data not shown). Sixty-two (62) of the 128 patients enrolled in this study have received at least three posterior juxtascleral administrations of anecortave acetate or placebo at 6-month intervals while 16 patients have received at least five such treatments. As of August 2002, 50 patients continue to be treated with masked study medication at 6-month intervals in this ongoing study. However, the efficacy results presented here are based upon a single (initial) administration of study medication.

There were no statistically significant differences in Baseline values among treatment groups with respect to age, sex, race, log MAR visual acuity, or lesion characteristics (Table 2). The original study design allowed treatment of predominantly classic subfoveal lesions only, but the protocol was later amended to also allow enrollment and treatment of minimally classic lesions. Of the 128 patients in this study, 80% (102 of 128) entered the study with predominantly classic lesions while 20% (26 of 128) entered with minimally classic lesions. A predominantly classic lesion is defined as one in which classic CNV occupies at least 50% of the area of the total lesion (defined for this study as angiographic evidence of neovascularization, associated contiguous areas of serous elevation of the RPE, elevated blocked fluorescence, blood and/or late staining). The Baseline patient characteristics in this study were generally similar to those reported for the Visudyne® TAP trial (2), except that more (80% vs. 40%) of the patients in the study reported here had predominantly classic lesions at Baseline.

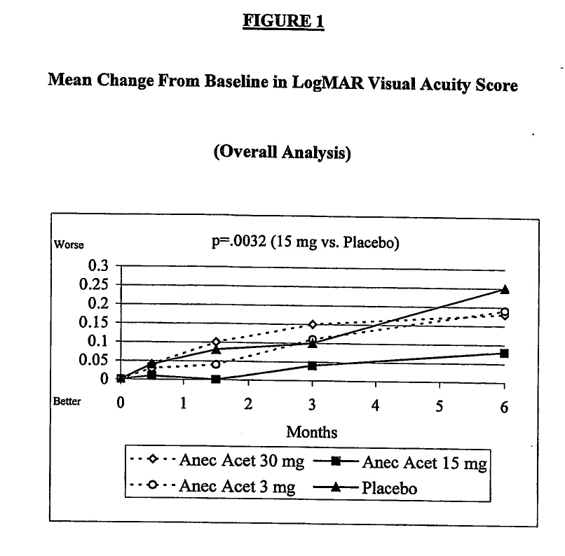

An interim analysis of all 128 patients was performed to evaluate mean change at Month 6 from Baseline values in log MAR visual acuity (FIG. 1). Anecortave acetate 15 mg is statistically superior to placebo treatment (p=0.0032) at Month 6. Trends also favor treatment with both anecortave acetate 30 mg and 3 mg over placebo treatment, although statistical significance is not achieved. Anecortave acetate 15 mg exhibits the greatest efficacy for stabilizing vision of the four groups.

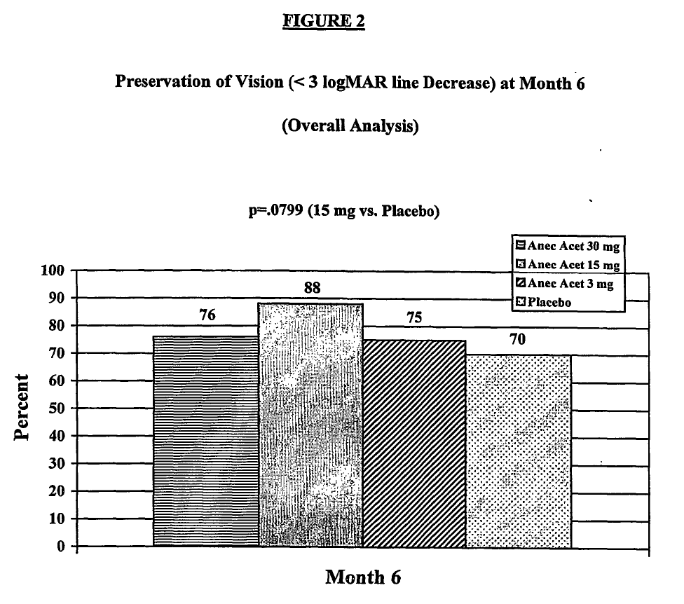

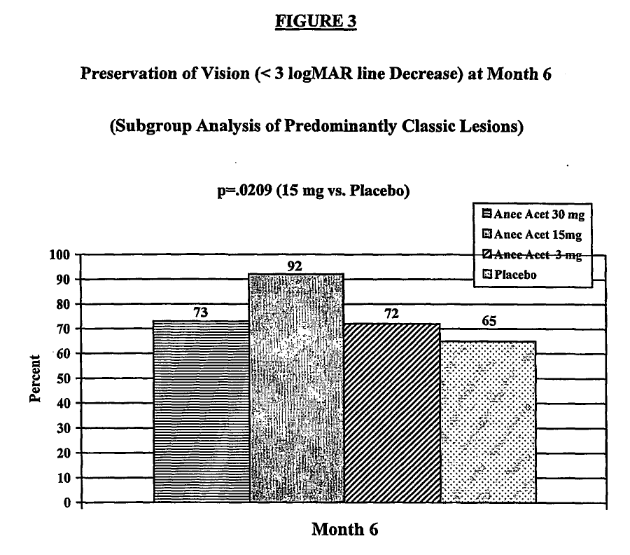

As a secondary visual outcome, the percentage of patients with preservation of vision at Month 6 was analyzed. Preservation (maintenance) of vision, defined as a decrease of less than three log MAR lines of visual acuity from Baseline values, is accepted as a clinically relevant measure of efficacy and has been used as a primary outcome variable in a previous report evaluating therapy for subfoveal AMD (2). The 6-month results of this analysis are presented in FIG. 2. There was greater preservation of vision at month six for patients treated with anecortave acetate 15 mg than for placebo, although the results did not achieve statistical significance at the p=0.05 level. Whereas 88% of patients treated with anecortave acetate 15 mg preserved vision at Month 6, only 70% of placebo-treated patients showed a similar positive visual outcome. However, as shown in FIG. 3, analysis of these data in the large subgroup of patients with a predominantly classic CNV lesion revealed a significant benefit favoring anecortave acetate 15 mg, with 92% of patients treated with anecortave acetate 15 mg preserving vision at Month 6 versus 65% of patients in the placebo group (p=0.0209). The efficacy of anecortave acetate 15 mg for preserving vision is further supported by data comparing clinically significant vision loss among treatment groups (Table 3). There is statistical superiority of anecortave acetate 15 mg at Month 6 compared with placebo (p=0.0224) for the prevention of severe vision loss (Table 4).

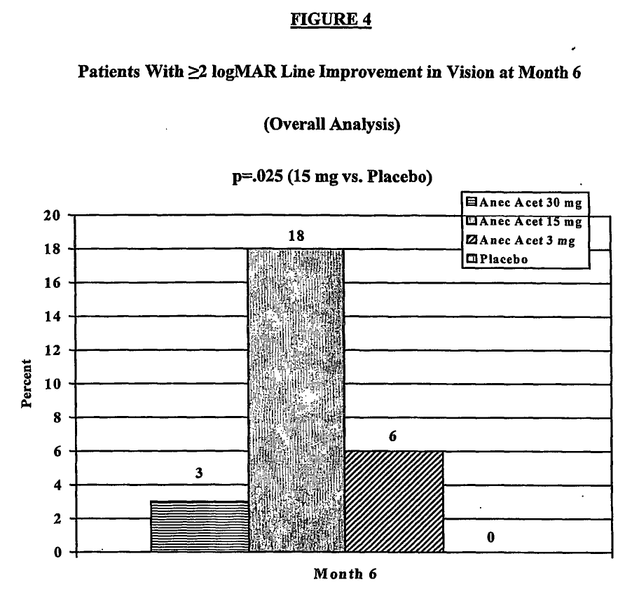

FIG. 4 shows the results of an analysis of the percentage of patients with an improvement at month six of at least 2 log MAR lines in visual acuity from Baseline values. Eighteen percent (18%) of patients treated with anecortave acetate 15 mg improved by at least 2 log MAR lines compared with 3% of patients in the 30 mg group, 6% in the 3 mg group, and 0% in the placebo group. The difference between anecortave acetate 15 mg and placebo was statistically significant (p=0.025).

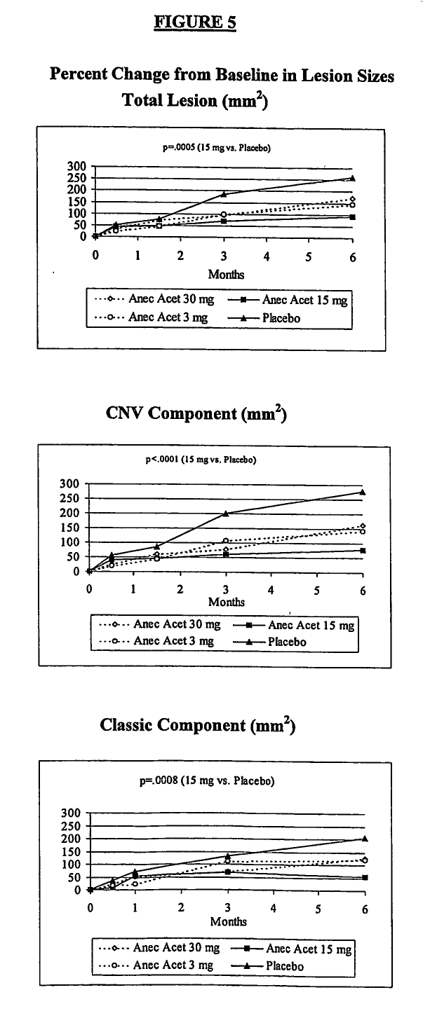

Because of preclinical data demonstrating anecortave acetate's angiostatic efficacy, CNV lesion changes from Baseline values in surface areas were analyzed. Total lesion areas, total CNV areas, and total classic CNV areas were measured and compared among treatment groups. While the average lesion size was similar among the treatment groups at Baseline, the variability within treatment groups reduced the sensitivity to demonstrate group differences when the groups were analyzed for mean change from Baseline values. Changes in these lesion characteristics were therefore analyzed as percent change from Baseline values, which proved to be a more sensitive measure for evaluating a population of lesions which ranged from 0.28 mm² to 33.25 mm² in total lesion areas at Baseline. As shown in FIG. 5, treatment with anecortave acetate 15 mg is statistically superior to placebo treatment for inhibition of total lesion surface area (p=0.0005), total CNV surface area (p=0.0001), and total classic CNV surface area (p=0.0008) at Month 6. In addition, there is a trend favoring anecortave acetate 30 mg and 3 mg treatment over placebo treatment for inhibition of lesion growth.

Following completion of the Month 6 visit by all patients, the accumulated safety data were evaluated by the Independent Safety Committee overseeing this study. Based upon this evaluation, no clinically relevant medication-related or administration-related safety concerns were identified. The most common ocular changes reported were changes in lens opacity using the Lens Opacity Classification System (LOCS) II, and included reports of nuclear color, nuclear opalescence, cortical, and posterior subcapsular changes. Cataracts are a common intercurrent disorder in this patient population, and the changes seen were documented in all treatment groups and in contralateral (untreated) eyes. The cataractous changes reported were described as mild and typically unrelated to treatment. The second most common ocular change, a decrease in vision (defined as a decrease of ≧4 log MAR lines from the previous visit), is also a common problem in this patient population. These vision decreases occurred in all treatment groups and in the contralateral eye. Other ocular changes (occurring with a frequency greater than 5%) were ptosis, ocular pain, subconjunctival hemorrhage, ocular pruritis, ocular burning/stinging, pupil disorders, foreign body sensation, ocular hyperemia, and abnormal vision. These changes were reported in all four treatment groups, in both treated eyes and contralateral eyes, were characterized as primarily mild, were generally not attributed to treatment, and were transient in nature. The single report of an IOP increase (≧10 mmHg) from Baseline occurred in a patient treated with anecortave acetate 30 mg and was attributed to intercurrent illness. Of the ocular changes reported, those most frequently attributed to study treatment were ptosis, ocular pain, subconjunctival hemorrhage, ocular pruritis, and ocular burning/stinging. These treatment-related events were mostly mild, transient, and seen within all four treatment groups.

The most common non-ocular changes from Baseline reported for this study were hypertension, peripheral edema, depression, and arthritis, none of which were attributed to treatment. No treatment-related changes in blood chemistry, hematology, or urinalysis were reported.

The data reported here are the result of an interim analysis of the first 6 months of clinical data from the ongoing study evaluating anecortave acetate as monotherapy for treatment of exudative AMD. This analysis demonstrates that a single posterior juxtascleral administration of anecortave acetate 15 mg is a safe and effective treatment for preserving or improving vision as well as preventing severe vision loss. These data also show that anecortave acetate inhibits lesion growth in patients with subfoveal CNV secondary to AMD. While there is a trend favoring a single administration of each of the three concentrations of anecortave acetate over placebo treatment, a single administration of anecortave acetate 15 mg is statistically superior to placebo for both functional and anatomical measures of clinical efficacy.

Anecortave acetate is an angiostatic agent developed for the inhibition of ocular neovascularization. Anecortave acetate is the result of specific chemical modification to the basic cortisol structure. These modifications have resulted in the creation of an angiostatic “cortisene,” which inhibits blood vessel growth, but does not produce glucocorticoid receptor-mediated steroidal side effects. Preclinical data show that anecortave acetate exhibits no measurable corticosteroid activity (8,9) and there is no clinical evidence of ocular corticosteroid side effects (such as elevated intraocular pressure or accelerated cataract progression) in the study reported here. Following an evaluation of safety data from patients with at least 6 months of anecortave acetate exposure, the Independent Safety Committee identified no clinically relevant drug-related or procedure-related safety issues.

Anecortave acetate is a unique angiostatic agent that upregulates plasminogen activator inhibitor 1 and inhibits both urokinase-like plasminogen activator and matrix metalloproteinase-3, two enzymes necessary for vascular endothelial cell migration during blood vessel growth (5,6). Preclinical data in models of corneal, retinal, and choroidal neovascularization support the efficacy of this agent for the inhibition of vessel growth (5,6,8-10).

The interim analysis of clinical data reported here demonstrate angiostatic efficacy at Month 6 following a single posterior juxtascleral administration, based on the masked evaluation of standardized fluorescein angiograms by DARC, the central reading center used for this study. This analysis shows that anecortave acetate 15 mg is statistically superior to placebo at Month 6 for inhibition of lesion growth. There was inhibition not only of total lesion growth but also of the CNV component and the classic CNV lesion component.

Analysis of the Month 6 data demonstrate a trend favoring anecortave acetate 15 mg over placebo treatment for preservation of vision in the overall analysis, and statistical superiority for preserving vision in the large subgroup of patients with predominantly classic lesions. Anecortave acetate 15 mg is also statistically superior to placebo for vision improvement, defined as an improvement of 2 or more lines of log MAR visual acuity. Conversely, a single treatment with anecortave acetate 15 mg inhibits both clinically significant vision loss and severe vision loss at Month 6 compared with placebo treatment.

The superiority of anecortave acetate 15 mg dose compared with placebo for stabilizing vision is demonstrated by the analysis of mean change at Month 6 from Baseline log MAR vision. Although the mean Baseline log MAR vision was very similar for the anecortave acetate 15 mg and placebo groups (0.73 vs. 0.76, respectively, or 20/100 Snellen equivalent), the vision outcome at Month 6 was distinctly different for these two treatment groups. After treatment with a single administration of anecortave acetate 15 mg, the mean vision changed by only 4 log MAR letters at Month 6, resulting in an average final log MAR value of 0.81 (20/125 Snellen equivalent). However, the placebo group over the same period worsened by more than 12 log MAR letters, resulting in an average final value of 1.01 (20/200 Snellen equivalent). This 2-line difference between groups in log MAR visual acuity is likely to have implications for the daily activities of a patient with subfoveal AMD.

All three doses of anecortave acetate have been shown here to be safe, and following a single administration there is a trend at Month 6 favoring the three doses for inhibition of lesion growth, for preservation of vision, and for prevention of severe vision loss. The clinical data reported here suggest the 15 mg dose is at or near the top of the biological dose-response curve for this molecule, and higher concentrations are not likely to be associated with greater efficacy in vivo. Alternatively, there may be differences in the formation and physical structure of the slow-release drug depot on the posterior scleral surface resulting from the different concentrations of drug suspension evaluated in this study which could in some way affect the absorption of the anecortave acetate into the overlying choroid and retina.

The clinical efficacy of anecortave acetate 15 mg compared with placebo for prevention of both clinically significant vision loss (defined as a loss of 15 or more log MAR letters) and severe vision loss (defined as a loss of 30 or more log MAR letters) at Month 6 is at least comparable to similar Month 6 data reported for the Visudyne® TAP study (2). In view of the consistent superiority of a single administration of anecortave acetate 15 mg compared with placebo treatment for preservation of vision and for inhibition of lesion growth, a pivotal study has been initiated to compare anecortave acetate 15 mg with Visudyne® PDT. This study is now enrolling patients, and includes 40-50 clinical sites in North America, Australia and the European Union.

It should be appreciated that anecortave acetate or its corresponding alcohol (4,9(11)-pregnadien-17α,21-diol-3,20 dione) can also be administered via a juxtascleral implant as described, e.g., in the following commonly owned patents and patent applications: U.S. Pat. No. 6,413,540B1; U.S. Pat. No. 6,416,777B1; WO/03/009784; and WO/03/009774. Juxtascleral administration via depot or by any other method provides for transcleral delivery of the drug. It can also be administered by an intravitreal injection or an implant, such as the one described in a co-pending U.S. application Ser. No. 10/385,791.

All patents and other references referred to herein are hereby incorporated by reference.

### REFERENCES

1. Seddon J M. Epidemiology of age-related macular degeneration. Retina, Ryan S J (ED.). St. Louis: Mosby, 2001; 1039-50.
2. Treatment of Age-related Macular Degeneration with Photodynamic Therapy (TAP) Study Group. Photodynamic therapy of subfoveal choroidal neovascularization in age-related macular degeneration with verteporfin—TAP Report 1. Arch. Ophthalmol. 1999; 117: 1329-45.
3. The EyeTech Study Group. Preclinical and phase IA clinical evaluation of an anti-VEGF pegylated aptamer (EYE001) for the treatment of exudative age-related macular degeneration. Retina 2002; 22: 143-52.
4. Krzystolik M G, Afshari M A, Adamis A P, et al. Prevention of experimental choroidal neovascularization with intravitreal anti-vascular endothelial growth factor antibody fragment. Arch. Ophthalmol. 2002; 120: 338-46.
5. DeFaller J M and Clark A F. A new pharmacological treatment for angiogenesis. In Pterygium, Taylor, H R (ED.) The Hague: Kugler Publications, 2000; 159-181.
6. Penn J S, Rajaratnam V S, Collier R J and Clark A F. The effect of an angiostatic steroid on neovascularization in a rat model of retinopathy of prematurity. Invest. Ophthalmol. Vis. Sci. 2001; 42: 283-90.
7. Casey R, Li W W. Factors Controlling Ocular Angiogenesis. Amer. J. Ophthalmol. 1997; 124: 521-529.
8. Clark A F. AL-3789: a novel ophthalmic angiostatic steroid. Exp. Opin. Invest. Drugs 1997; 6: 1867-77.
9. McNatt L G, Weimer L, Yanni J and Clark A F. Angiostatic activity of steroids in the chick embryo CAM and rabbit cornea models of neovascularization. J. Ocular Pharm. Therap. 1999; 15(5): 413-23.
10. BenEzra D, Griffin B W, Naftzir G, Sharif N A and Clark A F. Topical formulations of novel angiostatic steroids inhibit rabbit corneal neovascularization. Invest. Ophthalmol. Vis. Sci. 1997; 38: 1954-62.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

TABLE 1
Patient Inclusion and Exclusion Criteria

Inclusion Criteria
Patient must be willing to give and sign informed consent, able
to make the required study visits, and able to follow instructions.
Patient must be at least 50 years of age
Patients may be of any race and either sex. Female patients of
childbearing potential (those who are not postmenopausal or
surgically sterile) may participate only if they are not lactating,
if they have a negative pregnancy test at the Eligibility Visit
and prior to each of the three subsequent injections, and if they
agree to use adequate birth control methods (hormonal - oral,
implantable or injectable chemical contraceptives; mechanical -
spermicide in conjunction with a barrier such as condom or diaphragm;
IUD; or surgical sterilization of partner) to prevent pregnancy
throughout the 24 month study. Urine pregnancy tests will be
performed prior to each treatment, at each Month 3 post-treatment
Visit, and at the Exit Visit on all female patients of childbearing
potential. Male patients whose partner is of child bearing potential
should also use a reliable means to avoid fathering a child while
taking part in this study because the effect the study drug may
have on sex cells and what effect this would have on the development
of an unborn child is unknown.
Clinical diagnosis of exudative age-related macular degeneration
and a primary or recurrent subfoveal neovascular membrane which
does not meet MPS laser photocoagulation treatment guidelines and
which has the following characteristics as defined below:
A total lesion size of 12 disc areas or less,
AND
50% or more of the total lesion (defined as angiographic evidence
of neovascularization, associated contiguous areas of serous
elevation of the RPE, elevated blocked fluorescence, and/or late
staining) is choroidal neovascularization (CNV),
AND EITHER
The classic component of the total CNV is ≧50% of the total
choroidal neovascularization,
OR
The classic component of the total CNV is ≧0.75 disc areas
(1.6 mm²)
Clinical diagnosis of exudative age-related macular degeneration
and a primary or recurrent subfoveal neovascular membrane (with
the physical characteristics described above) which is eligible for
laser photocoagulation treatment by the most current MPS guidelines
but for which the patient has formally declined this approved
treatment in writing.
Best-corrected ETDRS visual acuity of 0.3 (20/40 Snellen) to
1.2 (20/320 Snellen) in the study eye at the Eligibility Visit.
The contralateral (“nonstudy”) eye must have a best-corrected
ETDRS visual acuity of 1.6 (20/800 Snellen) or better and clinical
evidence of macular degeneration (i.e., drusen, changes in the
retinal pigmented epithelium or signs of exudative disease or
disciform scarring).
Exclusion Criteria
Patient has a history of any medical condition which would
preclude scheduled study visits or completion of the study (i.e.,
unstable cardiovascular disease, unstable pulmonary disease or AIDS).
Patient has a history of ophthalmic disease in the study eye
(other than ARMD) that would likely compromise or during
follow-up could likely compromise the visual acuity of the study eye
(i.e., amblyopia, uncontrolled glaucoma with an IOP >30 mmHg,
ischemic optic neuropathy, clinically significant diabetic macular
edema, significant non-proliferative or proliferative diabetic
retinopathy, significant active uveitis).
Patient's screening fluorescein angiographic images and/or
indocyanine green angiographic images cannot be adequately
visualized by the investigator and the Digital Angiography
Reading Center.
Patient exhibits clinical signs of myopic retinopathy, or has a
refraction of >−8 diopter power in their current prescription.
Patient has had intraocular surgery in study eye less than
two (2) months prior to enrolling in the study.
Patient has a history of a previous experimental procedure for
the treatment of AMD in the study eye (excluding daily vitamin
and/or mineral therapy) other than laser photocoagulation treatment
for exudative ARMD in the study eye.
Patient has had insertion of a scleral buckle in the study eye
Use of any investigational drug or treatment related or
unrelated to ARMD within 30 days prior to receipt of study
medication, excluding daily vitamin and/or mineral therapy.
Patient has a known medical history of allergy or sensitivity
to the steroid family of drugs, or to fluorescein and/or
indocyanine green dyes that is clinically significant in the
investigator's opinion.
Patient has received radiation treatment (other than proton
beam radiation) or systemically administered anti-angiogenic
therapy for exudative ARMD in either eye. Patients with history
of proton beam treatment of fellow eye only may be enrolled in
this study.
Patient is on anticoagulant therapy, with the exception of
aspirin and antiplatelet therapy. Patient has a medical history
of a bleeding disorder.
Patient has clinical evidence of scleral thinning.

TABLE 2
Baseline Patient Characteristics

| | Anec Acet | | Anec Acet | | Anec Acet | | | | |
|---|---|---|---|---|---|---|---|---|---|
| | 30 mg | | 15 mg | | 3 mg | | Placebo | | |
| | (n = 33) | | (n = 33) | | (n = 32) | | (n = 30) | | |
| | N | % | N | % | N | % | N | % | P-value |
| Age (years) | | | | | | | | | |
| <65 | 4 | 12.1 | 5 | 15.2 | 1 | 3.1 | 0 | 0.0 | 0.3349 |
| 65-74 | 7 | 21.2 | 7 | 21.2 | 9 | 28.1 | 8 | 26.7 | |
| 75-84 | 19 | 57.6 | 17 | 51.5 | 15 | 46.9 | 19 | 63.3 | |
| 85-93 | 3 | 9.1 | 4 | 12.1 | 7 | 21.9 | 3 | 10.0 | |
| Female | 18 | 54.5 | 18 | 54.5 | 15 | 46.9 | 18 | 60.0 | 0.7781 |
| Caucasian | 33 | 100.0 | 33 | 100.0 | 32 | 100.0 | 30 | 100.0 | — |
| Total Lesion Classic | | | | | | | | | |
| Component* | | | | | | | | | |
| <50% Classic | 7 | 21.2 | 8 | 24.2 | 7 | 21.9 | 4 | 13.3 | 0.7333 |
| ≧50% Classic | 26 | 78.8 | 25 | 75.8 | 25 | 78.1 | 26 | 86.7 | |

| | | | | | |
|---|---|---|---|---|---|
| Age - Mean (SD) | 75.7 (7.5) | 75.8 (8.3) | 78.1 (7.5) | 78.3 (5.8) | 0.3193 |
| logMAR VA - Mean (SD) | 0.72 (0.26) | 0.73 (0.26) | 0.83 (0.24) | 0.76 (0.26) | 0.2859 |
| Total Lesion mm² - Mean (SD) | 8.6 (6.9) | 7.4 (6.6) | 9.0 (7.5) | 6.9 (5.7) | 0.5796 |
| CNV mm² - Mean (SD) | 7.4 (6.0) | 6.4 (5.5) | 7.9 (7.0) | 6.0 (5.2) | 0.5721 |
| Classic mm² - Mean (SD) | 5.7 (5.3) | 5.0 (5.0) | 5.3 (4.7) | 4.2 (4.2) | 0.6847 |

*Determined from the ratio of the size of the classic component to the size of the total lesion.

TABLE 3
Clinically Significant logMAR Vision Changes at Month 6

| | LogMAR Change | | | | | | | | | | | |
|---|---|---|---|---|---|---|---|---|---|---|---|---|
| | ≧2 Lines | | 1 Line | | No | | 1 Line | | 2 Lines | | ≧3 Lines | |
| | Improved | | Improved | | Change | | Worsened | | Worsened | | Worsened | |
| | N | % | N | % | N | % | N | % | N | % | N | % |
| Anec Acet 30 mg | 1 | 3.0 | 4 | 12.1 | 10 | 30.3 | 5 | 15.2 | 5 | 15.2 | 8 | 24.2 |
| Anec Acet 15 mg | 6 | 18.2 | 1 | 3.0 | 8 | 24.2 | 4 | 12.1 | 10 | 30.3 | 4 | 12.1 |
| Anec Acet 3 mg | 2 | 6.3 | 0 | 0 | 12 | 37.5 | 4 | 12.5 | 6 | 18.8 | 8 | 25.0 |
| Placebo | 0 | 0 | 2 | 6.7 | 11 | 36.7 | 4 | 13.3 | 4 | 13.3 | 9 | 30.0 |

TABLE 4
Severe Vision Loss (≧6 logMAR lines) at Month 6

| | Total | <6 Lines Worse | | ≧6 Lines Worse | |
|---|---|---|---|---|---|
| Treatment | N | N | % | N | % |
| Anec Acet 30 mg | 33 | 25 | 75.76 | 8 | 24.24 |
| Anec Acet 15 mg | 33 | 32 | 96.97 | 1 | 3.03 |
| Anec Acet 3 mg | 32 | 25 | 78.13 | 7 | 21.88 |
| Placebo | 30 | 23 | 76.67 | 7 | 23.33 |
| Total | 128 | 105 | 82.03 | 23 | 17.97 |

p = .0224, Anec Acet 15 mg vs. Placebo, Fisher's Exact Test

1. A method for the prevention of the loss of visual acuity associated with AMD, which comprises, administering a pharmaceutically effective amount of the compound anecortave acetate or its corresponding alcohol, wherein said administering is by a method selected from the group consisting of posterior juxtascleral injection, juxtascleral implant, intravitreal injection, or implant.

2. The method of claim 1, wherein the compound is administered as a juxtascleral depot.

3. The method of claim 2, wherein the depot comprises 3 mg-30 mg of compound.

4. The method of claim 3, wherein the depot comprises 15 mg of compound.

5. A method for maintaining visual acuity in a person suffering from AMD, which comprises administering a pharmaceutically effective amount of the compound anecortave acetate or its corresponding alcohol, wherein said administering is by a method selected from the group consisting of posterior juxtascleral injection, juxtascleral implant, intravitreal injection, or implant.

6. The method of claim 5, wherein the compound is administered as a juxtascleral depot.

7. The method of claim 6, wherein the depot comprises 3 mg-30 mg of compound.

8. The method of claim 7, wherein the depot comprises 15 mg of compound.

9. A method for the inhibition of lesion growth associated with AMD, which comprises administering a pharmaceutically effective amount of the compound anecortave acetate or its corresponding alcohol, wherein said administering is by a method selected from the group consisting of posterior juxtascleral injection, juxtascleral implant, intravitreal injection, or implant.

10. The method of claim 9, wherein the compound is administered as a juxtascleral depot.

11. The method of claim 10, wherein the depot comprises 3 mg-30 mg of compound.

12. The method of claim 11, wherein the depot comprises 15 mg of compound.

13. The method of any one of claims 1, 5, or 9, wherein the compound is administered in a juxtascleral implant.

14. The use of anecortave acetate or its corresponding alcohol for the preparation of a medicament for the prevention of the loss of visual acuity associated with AMD.

15. The use of anecortave acetate or its corresponding alcohol for the preparation of a medicament for maintaining visual acuity in a person suffering from AMD.

16. The use of anecortave acetate or its corresponding alcohol for the preparation of a medicament for the inhibition of lesion growth associated with AMD.

17. The use according to claims 14-16 wherein the preparation is administered as a juxtascleral depot.

18. The use according to claim 17 wherein the depot comprises 3 mg-30 mg of anecortave acetate or its corresponding alcohol.

19. The use according to claim 18 wherein the depot comprises 15 mg of anecortave acetate or its corresponding alcohol.

20. The use according to claims 14-16 wherein the preparation is administered as a juxtascleral implant.

21. A method for inhibiting blood vessel growth associated with AMD, said method comprising administering a pharmaceutically effective amount of the compound anecortave acetate or its accompanying alcohol, wherein the administering is by juxtascleral injection, intravitreal injection, juxtascleral implant, or other implant.

22. The method of claim 21, wherein the amount of compound administered is from 3 mg to 30 mg.

23. The method of claim 22, wherein the amount of compound administered is 15 mg.

24. The method of claim 9, wherein the lesion is a predominantly classic subfoveal lesion.

25. The method of claim 9, wherein the lesion is a minimally classic lesion.
